(19) United States
(12) Patent Application Publication
(10) Pub. No.: US 2023/0296746 A1
(43) Pub. Date: Sep. 21, 2023

(54) SYSTEMS AND METHODS FOR SCANNING

(71) Applicant: Exo Imaging, Inc., Redwood City, CA (US)

(72) Inventors: Seshadri Srinivasan, Redwood City, CA (US); Ruiying ZHANG, San Jose, CA (US); Biren PARMAR, San Jose, CA (US)

(21) Appl. No.: 17/696,542

(22) Filed: Mar. 16, 2022

(51) Int. Cl. G01S 7/52; A61B 8/08; A61B 8/06

(52) U.S. Cl. CPC G01S 7/52085; A61B 8/08; A61B 8/488; A61B 8/06; G01S 7/52055

(57) ABSTRACT

The present disclosure provides a method comprising: generating a map comprising an index of a plurality of scan tables; imaging a first target region using one or more scan tables selected from the plurality of scan tables; computing a parameter of interest based on one or more images obtained from the imaging of the first target region; and imaging the first target region using a subset of the one or more scan tables, which subset may be selected based on the computed parameter of interest.

### BACKGROUND

Non-intrusive imaging systems may be used to image internal tissue, bones, blood flow, or organs of human or animal body, or other objects of interest, such as a toy or a shipment package. Such systems and/or probes may require transmission of a signal into the body and the receiving of an emitted or reflected signal from the object or body part being imaged. In some cases, transducers or transceivers may be used to perform imaging, including imaging based on photo-acoustic and/or ultrasonic effects.

### SUMMARY

The present disclosure provides systems, devices, and methods for ultrasound imaging, including three-dimensional (3D) imaging based on ultrasound waves or other audio waves. Conventional ultrasound systems may employ a variety of scanning methods (also known as an event engine). For example, some scan methods may utilize simple event engines that execute one or more pre-determined scan tables. Other scan methods may utilize real-time in-line computations of various events or event sequences. In some cases, scan methods may comprise hybrid methods (combining pre-determined tables and in-line computations). These methods may involve overly simplistic or computationally expensive operations that are unable to efficiently adapt to changing or dynamic imaging conditions relating to the imaging environment or the operation or handling of ultrasound imaging systems/devices.

The present disclosure provides systems and methods that address the abovementioned shortcomings of conventional ultrasound systems. The systems and methods of the present disclosure may implement or utilize a dynamic scan-table selection that is based on predefined sets of operating conditions, also referred to herein as maps. These maps may be designed for improved frame-rate, optimized power consumption, enhanced image quality, real-time anatomy adaptive imaging, or any combinations of the above.

The scan tables disclosed herein may be used with any type of imaging system capable of optical and/or acoustic imaging. Examples of such compatible imaging systems may include, for instance, systems and devices configured for ultrasound imaging with matrix arrays having a plurality of (transducer) elements. The methods of the present disclosure may also utilize the various imaging systems or devices described herein to implement or perform ultrasound imaging or any other type of imaging based on optical or acoustic waves.

In one aspect, the present disclosure provides a method, comprising: (a) generating a map comprising an index of a plurality of scan tables; (b) imaging a first target region using one or more scan tables selected from the plurality of scan tables; (c) computing a parameter of interest based on one or more images obtained from the imaging of the first target region; and (d) imaging the first target region using a subset of the one or more scan tables, which subset may be selected based on the parameter of interest.

In some embodiments, the plurality of scan tables are ordered or grouped according to an imaging condition of interest. In some embodiments, the imaging condition of interest comprises a beam configuration, beam penetration, beam transmit power, imaging frame rate, imaging frequency, scanning line density, signal to noise ratio, or imaging resolution.

In some embodiments, the parameter of interest comprises a beam configuration, beam penetration, beam transmit power, imaging frame rate, imaging frequency, scanning line density, signal to noise ratio, or imaging resolution.

In some embodiments, the method may further comprise, prior to (a), loading a super-set of the plurality of scan tables onto a memory with instructions for executing steps (a)-(d).

In some embodiments, the method may further comprise persisting one or more image frames obtained using a first scan table and a second scan table to show a transition of imaging states or imaging parameters.

In some embodiments, the method may further comprise repeating steps (b)-(d) for a second target region. In some embodiments, the method may further comprise selecting a different scan table or a different set of scan tables for imaging of the second target region. In some embodiments, the second target region comprises a different anatomy than the first target region.

In some embodiments, the plurality of scan tables comprise a collection of events and associated scanning conditions or parameters for an imaging device. In some embodiments, the imaging device comprises an ultrasound or audio-acoustic imaging device. In some embodiments, the collection of events comprises one or more events comprising a description of a scanning condition or a scanning parameter for an imaging line. In some embodiments, the scanning condition or scanning parameter comprises a transmit pulse parameter, an aperture parameter, a delay parameter, a filter parameter, a decimation parameter, a line-spacing parameter, or a number of collinear transmits parameter.

In some embodiments, the map enables a dynamic selection of one or more optimal scan tables comprising one or more predetermined sets of operating conditions for one or more imaging events.

In some embodiments, the method may further comprise displaying one or more images of the first target region to a user or an operator of an imaging device used to capture the one or more images.

In another aspect, the present disclosure provides a system comprising: an imaging device; a memory comprising an index of a plurality of scan tables; and a processor, wherein the processor is configured to: image a first target region using one or more scan tables selected from the plurality of scan tables; compute a parameter of interest based on one or more images obtained from the imaging of the first target region; and image the first target region using a subset of the one or more scan tables, which subset is selected based on the computed parameter of interest.

In some embodiments, the plurality of scan tables may be ordered or grouped according to an imaging condition of interest. In some embodiments, the imaging condition of interest comprises a beam configuration, beam penetration, beam transmit power, imaging frame rate, imaging frequency, scanning line density, signal to noise ratio, or imaging resolution.

In some embodiments, the parameter of interest comprises a beam configuration, beam penetration, beam transmit power, imaging frame rate, imaging frequency, scanning line density, signal to noise ratio, or imaging resolution.

In some embodiments, the processor is further configured to persist one or more image frames obtained using a first scan table and a second scan table to show a transition of imaging states or imaging parameters over time.

In some embodiments, the processor is further configured to image a second target region. In some embodiments, the processor is further configured to select a different scan table or a different set of scan tables for imaging of the second target region. In some embodiments, the second target region comprises a different anatomy than the first target region.

In some embodiments, the plurality of scan tables comprise a collection of events and associated scanning conditions or parameters for the imaging device. In some embodiments, the imaging device comprises an ultrasound or audio-acoustic imaging device. In some embodiments, the collection of events comprises one or more events comprising a description of a scanning condition or a scanning parameter for an imaging line or one or more imaging operations performable using the imaging device. In some embodiments, the scanning condition or scanning parameter comprises a transmit pulse parameter, an aperture parameter, a delay parameter, a filter parameter, a decimation parameter, a line-spacing parameter, or a number of collinear transmits parameter.

In some embodiments, the processor is configured to dynamically select one or more optimal scan tables comprising one or more predetermined sets of operating conditions for one or more imaging events.

In some embodiments, the system may further comprise a display unit for displaying the one or more images of the first target region to a user or an operator of the imaging device.

In some embodiments, the plurality of scan tables are loaded onto the memory as a super-set of multiple scan tables.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present disclosure will be obtained by reference to the following detailed description that sets forth illustrative embodiments and the accompanying drawings.

FIGS. 1 and 2 illustrate various examples of imaging systems that can be configured to perform imaging using one or more scan tables, in accordance with some embodiments.

FIG. 3 illustrates an example of a scan table, in accordance with some embodiments.

FIG. 4 illustrates a system comprising an imaging device configured to utilize one or more scan tables, in accordance with some embodiments.

FIGS. 5A-5C schematically illustrate various scan tables that can be used to modulate the scanning condition of an imaging device, in accordance with some embodiments.

FIG. 6 shows an exemplary schematic diagram of an ultrasonic system, in accordance with some embodiments.

FIG. 7 shows an exemplary schematic diagram of an ultrasonic imaging system comprising a transducer with a pMUT array used to transmit and receive ultrasonic beams, in accordance with some embodiments.

FIG. 8 shows another exemplary diagram of an ultrasonic imaging system, in accordance with some embodiments.

FIG. 9 shows piezoelectric elements of a pMUT array, in accordance with some embodiments.

FIG. 10 shows a computer system that is programmed or otherwise configured to implement one or more of the methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Certain Definitions

Unless otherwise defined, all technical terms used herein have the same meaning as commonly understood by one of ordinary skill in the art to which the present subject matter belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

### Scan Tables

In one aspect, the present disclosure provides systems and methods for imaging. Such imaging may include, for example, ultrasound imaging. The imaging systems disclosed herein may be configured to implement one or more scan tables. The imaging methods disclosed herein may involve using one or more scan tables to perform imaging.

The one or more scan tables may comprise a description of an event. The event may comprise an imaging event or imaging operation as described in greater detail below. The event may comprise a scanning event. The one or more scan tables may further comprise a selection of different imaging parameters that correspond to the imaging event or imaging operation. The different imaging parameters may be optimized or tailored for a specific imaging event or imaging operation.

The one or more scan tables may be embodied or configured in a table format. In some cases, the table format may be configured for look-up operations. For instance, if a particular imaging event or imaging operation is detected (e.g., based on image analysis, sensor measurements, or user input), a computing device (e.g., a processor or a computer) may perform a look-up operation to identity or determine the optimal imaging parameters for the imaging operation or imaging event that is detected.

### Events

The one or more scan tables may comprise or represent a collection of events that determine the required or optimal scanning condition for an imaging frame. Each event may correspond to an imaging event or an imaging operation. Such imaging event or imaging operation may involve obtaining or capturing one or more images or videos or extracting one or more image frames from such images or videos. In some cases, the event may be defined by or associated with a specific imaging device, an imaging location or region or field of view, an imaging time, an imaging parameter (e.g., scan frequency), or an imaging vector (i.e., a vector or a series of vectors defining a path along which imaging can be performed). Each event may comprise a description of the scanning condition or parameter for an imaging line. In some cases, the imaging line may correspond to a column or row or other linear or non-linear series of pixels in an image. In some cases, the imaging line (also referred to herein as a scan line) may correspond to at least a portion of the imaging vector(s) along which imaging is performed.

In some cases (e.g., for a typical B-mode image), each event may correspond to or represent a scan-line from the left to the right of the image. In other cases (e.g., for a typical color-doppler image), there may be two types of scan-lines associated with an event, one for B-mode and another for Color-Doppler. The two types of scan-lines may coincide with one another or correspond to the same set of pixels.

As used herein, a scan line may correspond to and represent a portion of a frame representing an image. To form a frame, a transducer can focus and/or transmit waves (e.g., acoustic or pressure waves) from different piezoelectric elements to a particular focal point. The reflected signals collected by these piezoelectric elements can be received, delayed, weighted, and summed to form a scan line. The focal point of interest can be changed to different parts of the frame, and the process can be repeated until an entire frame comprising a plurality of scan lines is generated.

In some embodiments, the events may comprise one or more time-based events. The time-based events may be associated with imaging of a particular feature, imaging from a certain angle or location, imaging along a certain line or vector, and/or imaging using certain selected parameters. In some cases, the events may comprise two or more consecutive events in time. In other cases, the events may comprise two or more events that are non-consecutive. In any case, the two or more events may collectively enable imaging of one or more features in a target region or multiple target regions across a period of time. The period of time may be at least about 1 second, 10 seconds, 20 seconds, 30 seconds, 40 seconds, 50 seconds, 1 minute, 10 minutes, 20 minutes, 30 minutes, 40 minutes, 50 minutes, 1 hour, or more.

In some cases, the events may be detected automatically based on one or more images taken using the imaging device. For instance, the images taken by the imaging device may be analyzed using an image analysis algorithm to determine the scanning event or scanning operation that is taking place or that the user wishes to perform. In other cases, the events may be detected automatically based on one or more sensor readings or measurements (e.g., from accelerometers or motion sensors operatively coupled to the imaging device or any of the scanning systems described herein). Alternatively, the user of the imaging device may manually input the desired scanning event or scanning operation, and one or more appropriate scan tables may be utilized to implement the corresponding optimal scanning conditions.

In some embodiments, changes in scanning events over time may be detected (e.g., using the image analysis methods or sensor-based methods described above). As changes in scanning events are detected, the appropriate scan tables may be automatically loaded or implemented to enable live adaption and selection of optimal scanning parameters.

In some embodiments, the one or more scan tables may collectively map or correlate events or scan lines across one or more target regions to optimal imaging parameters for the one or more target regions. In some cases, multiple surfaces or terrains of a target region may be mapped to corresponding events and/or scanning conditions/parameters using the one or more scan tables described herein. As described in greater detail below, the one or more scan tables may be aggregated or combined into a super-set. The super-set may comprise multiple scan tables each comprising a list of optimal imaging parameters and associated values for different types of events. The optimal imaging parameters and associated values may be mapped to different types of events (including imaging events or operations). The maps may be designed for improved frame-rate, optimized power consumption, enhanced image quality, anatomy adaptive imaging, or any combinations of the above.

### Scanning Condition/Parameter

As described above, the one or more scan tables may comprise a description of an event and associated scanning conditions or parameters for the event. The scanning conditions or parameters may be set or adjusted differently for different imaging operations, different users, and/or different imaging devices.

In some embodiments, the scanning condition or parameter may comprise, for example, the transmit pulse, pulse width, pulse power, pulse repetition interval, pulse order, pulse timing, pulse cycle, transmit frequency, pulse start time, pulse end time, pulse duration, or pulse type (e.g., unipolar pulses, multi-state bipolar pulses), or any other property associated with a pulse that is transmittable by the imaging device. The pulse may comprise an audio or ultrasound signal or wave. In some embodiments, the scanning condition or parameter may comprise pulse frequency, pulse wavelength, phase delay, or phase differences between two or more pulses.

In some embodiments, the scanning condition or parameter may comprise transmit focus depth, or penetration depth. In some embodiments, the scanning condition or parameter may comprise a number of beams, a type of beam (pulsed vs continuous wave), a beam size, a beam geometry (e.g., beam origin, beam directionality, beam elevation/azimuth, or any other property or characteristic of a beam that can be generated and transmitted by the imaging device). The beam may comprise an audio or ultrasound signal or wave.

In some embodiments, the scanning condition or parameter may comprise a scanning mode. The scanning mode may comprise, for example, 2D imaging or 3D imaging. In some embodiments, the scanning condition or parameter may comprise scanning speed, scanning direction, or scanning pattern.

In some embodiments, the scanning condition or parameter may comprise the aperture (e.g., aperture size or aperture shape). In some embodiments, the scanning condition or parameter may comprise the type of filter used, transmit delay, gain, weighting or apodization of signals transmitted or received by individual transducer elements, decimation, line-spacing, or a number of collinear transmits per unit time or per unit area.

### Flexible Scan Tables

The one or more scan tables may be flexible, dynamic, and/or adjustable. Such flexibility may allow for automatic real-time switching or updating of scan tables in real time and on the fly as imaging (e.g., ultrasound imaging) is being performed.

In some cases, the one or more scan tables may be changed, interchanged, or dynamically selected in real time based on one or more predefined sets of operating conditions, also referred to herein as maps. In some cases, the one or more scan tables and/or the one or more maps corresponding to various predefined sets of operating conditions may be dynamically adjusted or updated in real time based on (1) external inputs from a user, (2) changes in operating condition or image quality, or (3) other factors relating to imaging performance or operator preference.

### Methods

In one aspect, the present disclosure provides a method for imaging. The method may comprise loading one or more scan tables for satisfying different operating conditions. The one or more scan tables may comprise, in some instances, a super-set of multiple scan tables. The scan tables may be optimized or customized for different imaging applications, different imaging conditions, or different operators.

The one or more scan tables may be configured for imaging under different operating conditions. The operating conditions may comprise, for example, penetration, frame-rate, power consumption, imaging resolution, or transmit power. In some cases, the operating conditions may comprise one or more parameters corresponding to an operation of an imaging device (e.g., an ultrasound imaging device).

In some embodiments, the one or more parameters may comprise, for example, the transmit pulse, pulse width, pulse power, pulse repetition interval, pulse order, pulse timing, pulse cycle, transmit frequency, pulse start time, pulse end time, pulse duration, or pulse type (e.g., unipolar pulses, multi-state bipolar pulses), or any other property associated with a pulse that is transmittable by the imaging device. The pulse may comprise an audio or ultrasound signal or wave. In some embodiments, the one or more parameters may comprise pulse frequency, pulse wavelength, phase delay, or phase differences between two or more pulses. In some embodiments, the one or more parameters may comprise transmit focus depth, or penetration depth. In some embodiments, the one or more parameters may comprise a number of beams, a type of beam (pulsed vs continuous wave), a beam size, a beam geometry (e.g., beam origin, beam directionality, beam elevation/azimuth, or any other property or characteristic of a beam that can be generated and transmitted by the imaging device). The beam may comprise an audio or ultrasound signal or wave. In some embodiments, the one or more parameters may comprise a scanning mode. The scanning mode may comprise, for example, 2D imaging or 3D imaging. In some embodiments, the one or more parameters may comprise scanning speed, scanning direction, or scanning pattern. In some embodiments, the one or more parameters may comprise the aperture (e.g., aperture size or aperture shape). In some embodiments, the one or more parameters may comprise the type of filter used, transmit delay, gain, weighting or apodization of signals transmitted or received by individual transducer elements, decimation, line-spacing, or a number of collinear transmits per unit time or per unit area.

In some embodiments, the method may comprise creating a map that represents an index of multiple scan tables. The multiple scan tables may be ordered according to the conditions of interest. For instance, a first scan table corresponding to a first condition of interest may be ordered or prioritized before a second scan table corresponding to a second condition of interest. The first condition of interest may be different than the second condition of interest. The conditions of interest may change over time and for different imaging applications or different operators. In some cases, the multiple scan tables may be ordered or re-ordered based on the current imaging application or the preferences of a user or operator of the imaging device.

In some embodiments, the method may comprise selecting a preferred map of the scan table for a default imaging condition. Selection of the preferred map may be performed manually by a user or an operator. Alternatively, selection of the preferred map may be performed automatically by a computer or an algorithm. In some cases, the preferred map of the scan table may be assigned to a user-defined preset imaging mode.

In some embodiments, the method may comprise imaging a target using the selected scan-table. The selected scan-table may be used to automatically perform and/or control imaging of the target. In some cases, the selected scan-table may be used for a first imaging operation. Once the first imaging operation is completed, a different scan-table may be used or selected for a second imaging operation. In some cases, the selected scan-table may be used for multiple imaging operations. The multiple imaging operations may be performed during contiguous time intervals. Alternatively, the multiple imaging operations may not or need not be performed during contiguous time intervals.

In some embodiments, the method may comprise determining or computing one or more parameters of interest from one or more images obtained for a target. The one or more parameters of interest may comprise, for example, penetration depth, signal to noise ratio (SNR), imaging resolution (which may include spatial resolution and/or temporal resolution), imaging frame rate, or transmit power.

In some embodiments, the method may further comprise choosing an appropriate set or subset of scan tables based on the parameter of interest. For example, the super-set of scan tables may comprise a first scan table that is optimal for imaging based on a first parameter of interest (e.g., a desired frame rate) and a second scan table that is optimal for imaging based on a second parameter of interest (e.g., signal to noise ratio). If a user or operator is interested in imaging based on the first parameter of interest (e.g., a specific frame rate), the first scan table may be selected and used to control subsequent imaging operations. If the user or operator is later interested in imaging based on the second parameter of interest (e.g., a certain acceptable signal to noise ratio), the second scan table may be selected and used to control subsequent imaging operations.

In any of the embodiments described herein, the super-set of multiple scan tables may comprise a plurality of scan tables corresponding to (i) a same parameter of interest or (ii) different parameters of interest. In some cases, multiple scan tables may be associated with the same or similar parameter or type of parameter. In some instances, a first scan table may be associated with a first set of parameters or conditions for a particular imaging operation, and a second scan table may be associated with a second set of parameters or conditions for another imaging operation, wherein the first set of parameters or conditions and the second set of parameters or conditions are the same type or a similar type of parameter or condition. For example, a first scan table may be associated with a first imaging frame rate and a second scan table may be associated with a second imaging frame rate. In some cases, the first scan table may be selected for a first imaging operation and the second scan table may be selected for a second imaging operation. The first imaging operation and the second imaging operation may both correspond to imaging of a same target or a same or similar feature of the target. Alternatively, the imaging operation and the second imaging operation may both correspond to imaging of different targets or different features of the same target.

In some embodiments, the one or more selected scan tables (e.g., scan tables selected to optimize one or more parameters of interest) may be used for one or more imaging operations. In some cases, during or after the one or more imaging operations, additional or alternative scan tables may be selected for imaging.

In some embodiments, the method may comprise persisting one or more frames from a first scan table selected at a first point in time and a second scan table selected at a second point in time. Persisting the one or more frames from the first scan table and the second scan table may help to show or visualize a gradual transition of the imaging states or conditions over time (e.g., during imaging). The transition between imaging states may help to guide or inform a user to further optimize the imaging operations (e.g., by selecting additional or alternative scan tables that can enable imaging tailored to a particular use case or a particular set of operator or user preferences).

### Examples

In one aspect, the present disclosure provides a method of imaging. The method may comprise performing imaging using an imaging device (e.g., an ultrasound imaging device). The method may further comprise computing image signal levels as a function of depth for the current imaging operation. The method may further comprise comparing the signal levels and associated penetration depth against the expected penetration for the current imaging preset. The method may further comprise choosing a lower transmit frequency to improve the penetration of imaging, based on the comparison. In some embodiments, the method may comprise selecting an appropriate scan table that can aid in optimizing the penetration depth (e.g., to better visualize or image features of interest that are located or positioned at a certain depth or distance relative to the imaging device).

In another aspect, the present disclosure provides a method of imaging. The method may comprise performing imaging using an imaging device (e.g., an ultrasound imaging device). The method may further comprise computing or determining the frame-rate of imaging using multiple image frames and the timings at which the image frames are captured or obtained. The method may further comprise selecting a different transmit line-density setting to optimize or improve the frame rate accordingly. In some embodiments, the method may comprise persisting the new and previous frames to gradually change the imaging state. Such a gradual change may indicate affirmatively to the user that the frame rate is being adjusted or has been adjusted. In some embodiments, the method may comprise selecting an appropriate scan table that can aid in improving frame rates (e.g., to improve or enhance tracking of changes or movement of features in the images obtained using the imaging device).

In another aspect, the present disclosure provides a method of imaging. The method may comprise performing imaging using an imaging device (e.g., an ultrasound imaging device). The method may further comprise determining the current operating power based on electrical current, temperature, and/or battery levels for the imaging device, and optimizing an operation of the imaging device accordingly. In some embodiments, the method may further comprise selecting from multiple beam operating conditions (e.g., 5-parallel beam or 3-parallel beam or single beam operating modes) based on the current operating condition, the desired operating condition, the current operating power (i.e., current power draw over time), and/or the desired operating power (i.e., desired power draw over time). In some embodiments, the method may comprise selecting an appropriate scan table that can aid in lowering operating power (e.g., to minimize damage to certain surrounding regions or neighboring objects/features that are sensitive to one or more signals emitted by the imaging device, or to extend the battery life of the imaging device).

In another aspect, the present disclosure provides a method of imaging. The method may comprise performing imaging using an imaging device (e.g., an ultrasound imaging device). The method may further comprise selecting one or more lines or regions within a current image. The method may further comprise computing the axial resolution based on the spectrum as well as point/specular targets from those lines. The method may further comprise computing the lateral resolution from the point targets. The method may further comprise choosing a desired imaging frequency (e.g., a higher imaging frequency) and/or a desired imaging bandwidth (e.g., a higher imaging bandwidth) based on the desired imaging resolution, in order to optimize spatial resolution. In some embodiments, the method may further comprise adjusting one or more imaging focus location(s) in order to optimize the image quality (e.g., sharpness, focus, contrast, etc.) and/or spatial resolution. In some embodiments, the method may comprise selecting an appropriate scan table that can aid in optimizing spatial resolution, imaging frequency, imaging bandwidth, and/or image focus for subsequent imaging operations.

In another aspect, the present disclosure provides a method of imaging. The method may comprise performing imaging using an imaging device (e.g., an ultrasound imaging device). The method may further comprise identifying one or more parameters of interest such as, for example, signal-to-noise ratio, spatial resolution of speckle or specular targets, or motion of targets, from one or more images acquired using an imaging device. The method may further comprise choosing a set of operating conditions such as frequency, focus, and/or line-density based on the target of interest. In some embodiments, the method may comprise selecting one or more appropriate scan tables for different types of anatomy to aid in anatomy adaptive imaging. For example, a first scan table may be selected to image a first anatomy, and a second scan table may be selected for imaging of a second anatomy that is different than the first anatomy. In another example, when an imaging device is used to image a heart and the operator moves the imaging device to image a liver, a computer can be used to automatically switch scan tables. In some cases, when imaging switches to different features of interest, the computer can also be configured to automatically switch scan tables to optimize imaging for the different features of interest.

### Imaging Applications

The scan tables described herein may be used to provide numerous benefits for imaging applications, including, for example, improved penetration, improved frame rate, lower operating power, improved spatial resolution, and anatomy adaptive imaging. These benefits may be realized individually or in combination with each other, depending on the use case.

In some cases, the scan tables may improve penetration. In some cases, the systems and methods of the present disclosure may be implemented to compute signal levels as a function of depth for the current imaging. The signal levels may be compared to expected penetration for the current preset. Based on such comparison, the systems may choose a lower transmit frequency to improve the penetration of imaging.

In some cases, the scan tables may improve frame rate. In some cases, the systems and methods of the present disclosure may be implemented to compute the frame-rate of imaging using multiple frames. A lower transmit line-density may be selected, and the new and previous frames may persist to gradually change the imaging state.

In some cases, the scan tables may lower operating power. Current, temperature, and battery level monitors may be used to determine the current operating power. Based on the operating condition, one of multiple parallel beam operating conditions may be selected. The multiple parallel beam operating conditions may comprise, for example, a single beam operating condition or an n-parallel beam operating condition, where n ranges from 1 to 100 or more.

In some cases, the scan tables may improve spatial resolution. In some cases, one or more lines may be selected from a current image. The axial resolution may be computed based on the wavelength spectrum as well as point/specular targets from those lines. The lateral resolution may be computed from the point targets. Thereafter, a higher imaging frequency and/or a higher imaging bandwidth may be chosen based on the desired resolution. In some cases, one or more imaging focus location(s) may be adjusted.

In some cases, the scan tables may enable anatomy adaptive imaging. In some cases, the systems and methods of the present disclosure may be used to identify one or more parameters of interest. The one or more parameters of interest may comprise, for example, signal-to-noise ratio, spatial resolution of speckle or specular targets, and/or motion of targets. The one or more parameters of interest may correspond to a quality, characteristic, or feature of one or more images that are obtained or can be obtained using the presently disclosed systems and methods. In some cases, one or more optimal operating conditions may be selected based on the target of interest. The optimal operating conditions may comprise, for example, frequency, focus, and/or line-density.

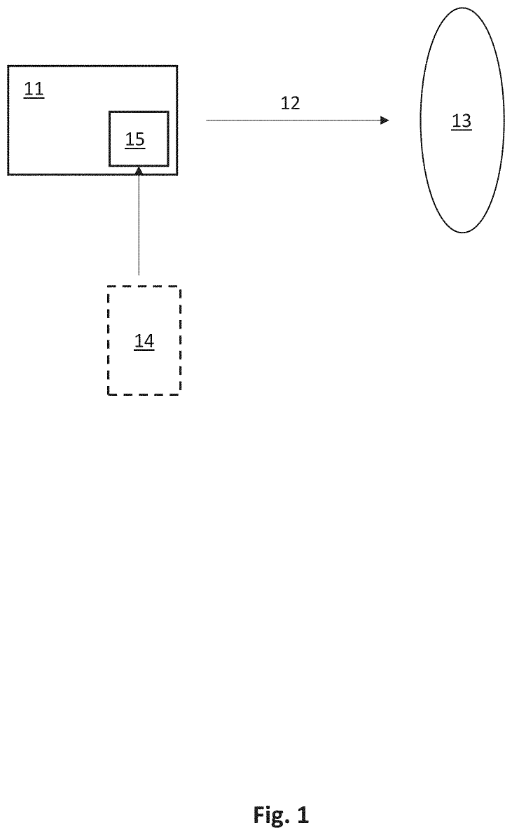

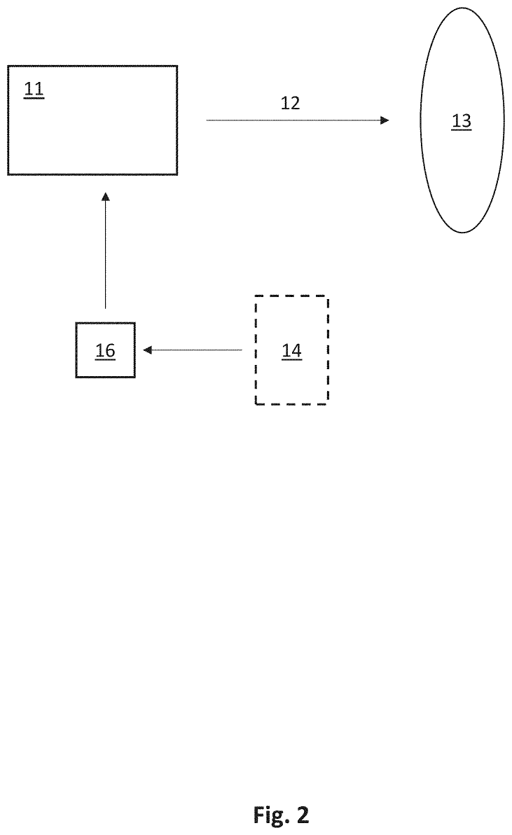

FIG. 1 illustrates an exemplary system 11 that may be used for imaging (e.g., ultrasound imaging) of a target region 13. The system 11 may comprise an imaging device (e.g., any of the imaging devices shown or described herein). The imaging device may be configured to transmit and receive one or more signals 12. The one or more signals 12 may comprise signals that are transmitted to and/or reflected from the target region 13. In some cases, an operation or scanning condition of the imaging device may be controlled or adjusted using one or more scan tables 14. The one or more scan tables 14 may be provided to a memory 15 of the system 11 or the imaging device (e.g., via a wired connector or a wireless connection). Alternatively, the scan tables 14 may be provided to a controller 16 that is operatively coupled to or in communication with the system 11 or the imaging device, as shown in FIG. 2. In such cases, the controller 16 may be configured to control the operation or scanning condition of the imaging device. In some cases, the controller 16 may be integrated with the system 11 or the imaging device. In other cases, the controller 16 may be remote from the system 11 or the imaging device.

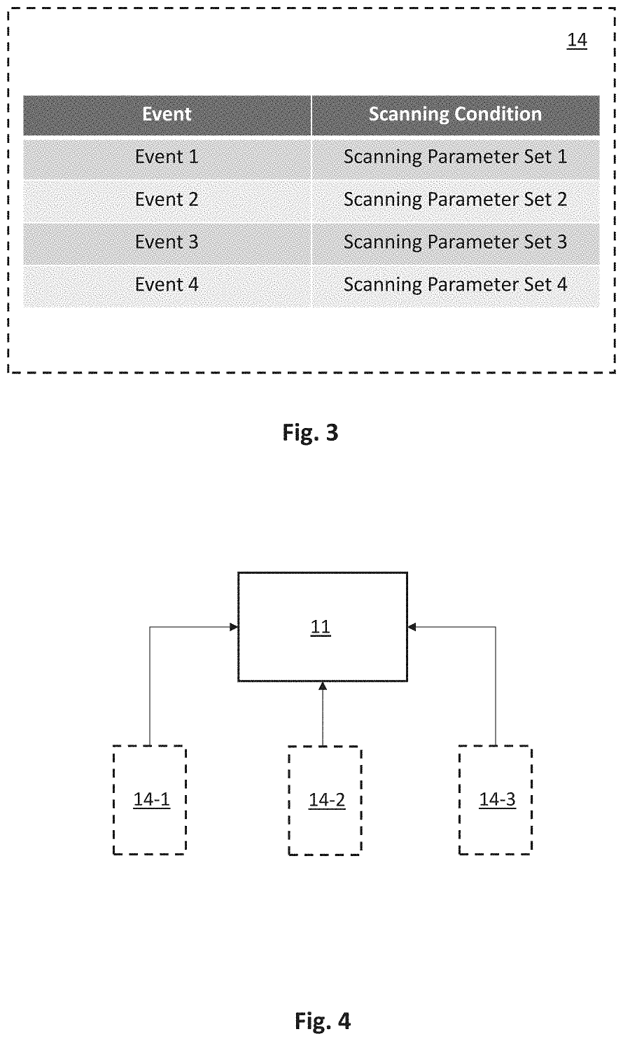

FIG. 3 illustrates an example of a scan table 14. The scan table may comprise one or more events and one or more scanning conditions associated with the one or more events. The one or more scanning conditions may comprise one or more scanning parameters (or sets of scanning parameters) that dictate or control an operation of the imaging device (e.g., a scanning operation performable using the imaging device).

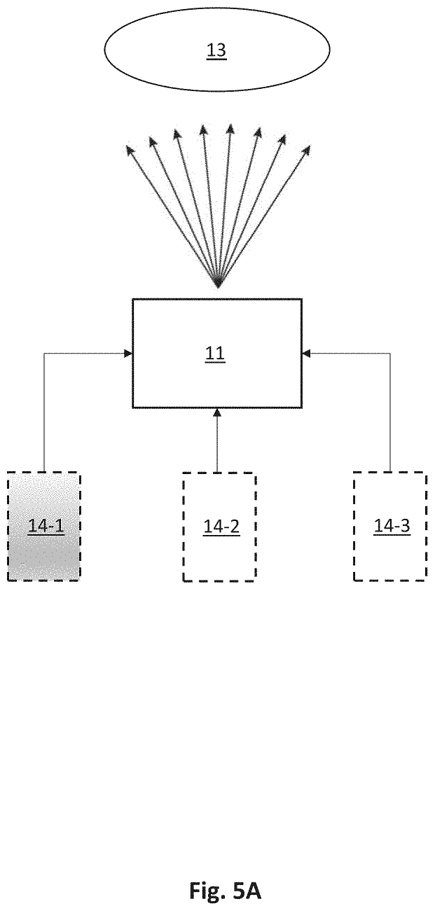

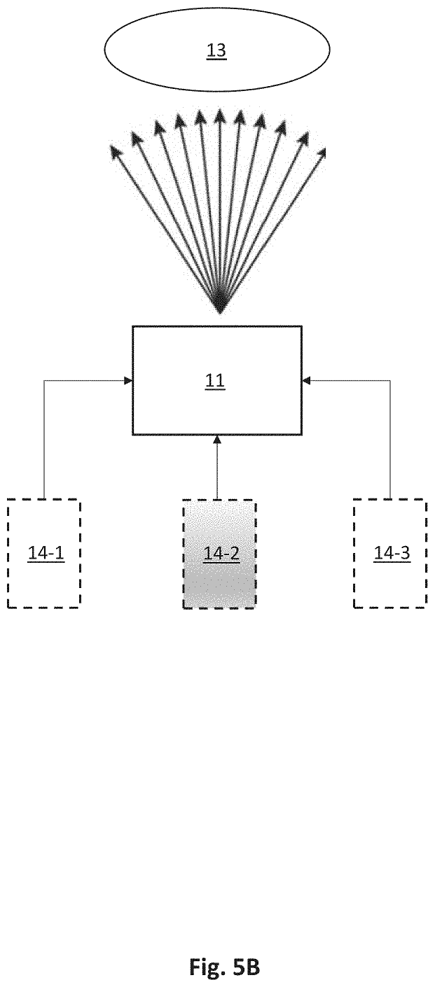

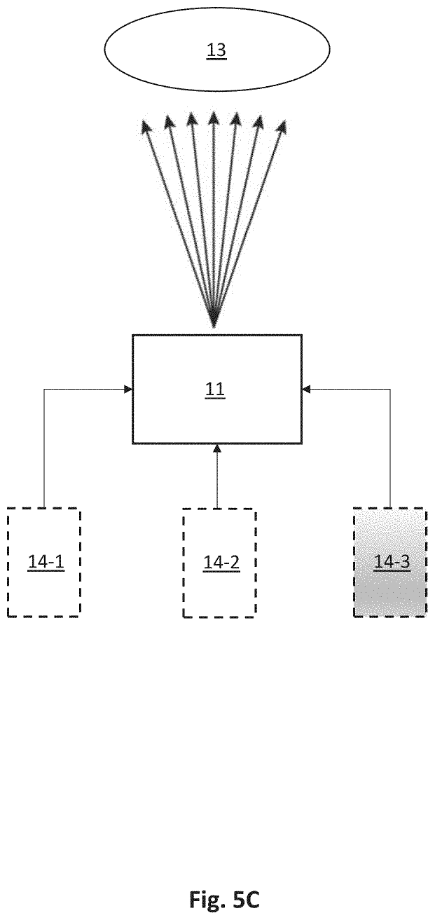

FIG. 4 illustrates a system 11 comprising an imaging device. The imaging device may comprise any imaging device described herein. The system 11 may be configured to utilize one or more scan tables 14-1, 14-2, 14-3, etc. to control an operation of the imaging device. The one or more scan tables 14-1, 14-2, 14-3 may be manually selected by a user or automatically selected by a computer to enhance or optimize an imaging performance of the imaging device. As shown in FIGS. 5A-5C, each of the one or more scan tables 14-1, 14-2, 14-3 may modulate the scanning condition of the imaging device differently. For example, the one or more scan tables 14-1, 14-2, 14-3 may be implemented to change the number of scan lines and/or the density of the scan lines used to image a target region 13 or a portion thereof.

In some embodiments, the scan tables described herein may be preloaded on a memory of the imaging device. In other embodiments, the scan tables may be stored on a memory of a computer that is remote from the imaging device. The computer may be operatively coupled to the imaging device to control or modulate an operation of the imaging device based on the scanning event and the scanning conditions defined in the scan tables.

In some embodiments, the scan tables may be transmitted from a mobile device of the user or operator of the imaging device to the imaging device. In some embodiments, the scan tables may be transmitted from a computer of the user or operator of the imaging device to the imaging device. The scan tables may be transmitted to the mobile device via a physical connection or a wireless connection.

### Ultrasound Imager/Imaging Probe

The scan tables described herein may be used compatibly with any type of imaging device, including optical and acoustic imaging devices. In some cases, the optical imaging devices may comprise cameras, CMOS sensors, CCD sensors, or any other type of sensor configured for imaging based on optical signals. In some cases, the acoustic imaging devices may comprise ultrasound imaging devices. In some non-limiting embodiments, the imaging devices may be configured as handheld ultrasound imaging probes.

Ultrasound imaging (sonography) uses high-frequency sound waves to view inside the body. Because ultrasound images are captured in real-time, they can show movement of the body's internal organs as well as blood flowing through the blood vessels. The sound waves can also be used to create and display images of internal body structures such as tendons, muscles, joints, blood vessels, and internal organs.

To perform imaging, the imaging device transmits a signal into the body and receives a reflected signal from the body part being imaged. Types of imaging devices include transducers, which may also be referred to as transceivers or imagers, and which may be based on either photo-acoustic or ultrasonic effects. Such transducers can be used for imaging as well as other applications. For example, transducers can be used in medical imaging to view anatomy of tissue or other organs in a body. Transducers can also be used in industrial applications such as materials testing or therapeutic applications such as local tissue heating of HIFU based surgery. When imaging a target and measuring movement of the target, such as flow velocity and direction blood, Doppler measurements techniques are used. Doppler techniques are also applicable for industrial applications to measure flow rates, such as fluid or gas flow in pipes. The Doppler measurements may be based on the difference between transmitted and reflected wave frequencies due to relative motion between the source and the object. The frequency shift is proportional to the movement speed between the transducer and the object. This effect is exploited in ultrasound imaging to determine blood flow velocity and direction.

In some embodiments, the transducer elements described herein (e.g., pMUT elements, cMUT elements, etc.) may be interchangeably referred to as transceiver elements. In some cases, the transducer elements described herein may comprise piezoelectric elements or piezo elements. In some embodiments, the transducer elements described herein may include one or more of: a substrate, a membrane suspending from the substrate; a bottom electrode disposed on the membrane; a piezoelectric layer disposed on the bottom electrode; and one or more top electrodes disposed on the piezoelectric layer.

For ultrasound imaging, transducers can be used to transmit an ultrasonic beam towards the target to be imaged. A reflected waveform is received by the transducer, converted to an electrical signal and with further signal processing, an image is created. Velocity and direction of flow may be measured using an array of micro-machined ultrasonic transducers (MUTs).

The ultrasound devices disclosed herein may be configured for B-mode imaging. B-mode imaging for anatomy is a two-dimensional ultrasound image display composed of dots representing the ultrasound echoes. The brightness of each dot is determined by the amplitude of the returned echo signal. This allows for visualization and quantification of anatomical structures, as well as for the visualization of diagnostic and therapeutic procedures. Usually, the B-mode image bears a close resemblance to the actual anatomy of a cutout view in the same plane. In B-mode imaging, a transducer is first placed in a transmit mode and then placed in receive mode to receive echoes from the target. The echoes are signal processed into anatomy images. The transducer elements are programmable such that they can be either in transmit mode or in receive mode, but not simultaneously.

The ultrasound devices disclosed herein may be configured for color Doppler imaging. The use of color flow Doppler, color Doppler imaging, or simply color Doppler allows the visualization of flow direction and velocity for blood in an artery or vein within a user defined area. A region of interest is defined, and the Doppler shifts of returning ultrasound waves are color-coded based on average velocity and direction. Sometimes these images are overlapped (co-imaged) with anatomy images in B-mode scan to present a more intuitive feel of flow relative to anatomy being viewed. Doppler imaging can also be PW Doppler so that the range and velocity of flow is determined, but maximum flow rate is dependent on pulse repetition frequency used, otherwise images are aliased making higher velocities look like lower velocities. Doppler shift can be measured from an ensemble of waves received to measure flow velocity using PW mode of Doppler imaging. CW Doppler is a continuous imaging technique where aliasing is avoided through continuous transmitting from one transducer element while receiving echoes from another transducer element. In a programmable instrument, both pulsed and continuous techniques can be implemented as discussed later. PW and Color Doppler may use a selected number of elements in an array. First, the elements are placed in a transmit mode and after echoes have returned, the elements are placed in a receive mode where the received signal is processed for Doppler signal imaging. For CW Doppler, at least two different elements are utilized, where each element is in transmit mode while the other element is in receive mode continuously.

The ultrasound imaging devices disclosed herein may be configured for two-dimensional (2D) imaging and/or three-dimensional (3D) imaging. In some embodiments, the ultrasound imaging devices may utilize one or more arrays of MEMS (micro-electromechanical system) ultrasound transducers such as pMUTs (piezoelectric micromachined ultrasound transducers) and/or cMUTs (capacitive micromachined ultrasound transducers). The micromachined ultrasonic transducers (MUTs) may be arranged in a lateral array. In some cases, the MUTs may be arranged in a regular or symmetric configuration. In other cases, the MUTs may be arranged in a staggered or asymmetric configuration. In some cases, the transducer elements may be arranged in a rectangular grid. In other cases, the elements can be arranged in a circular configuration, a rhombus (equilateral parallelogram), a hexagon, an annular shape, or an arbitrary grid, for example. The arrays can be on a curved surface as well as planar arrays.

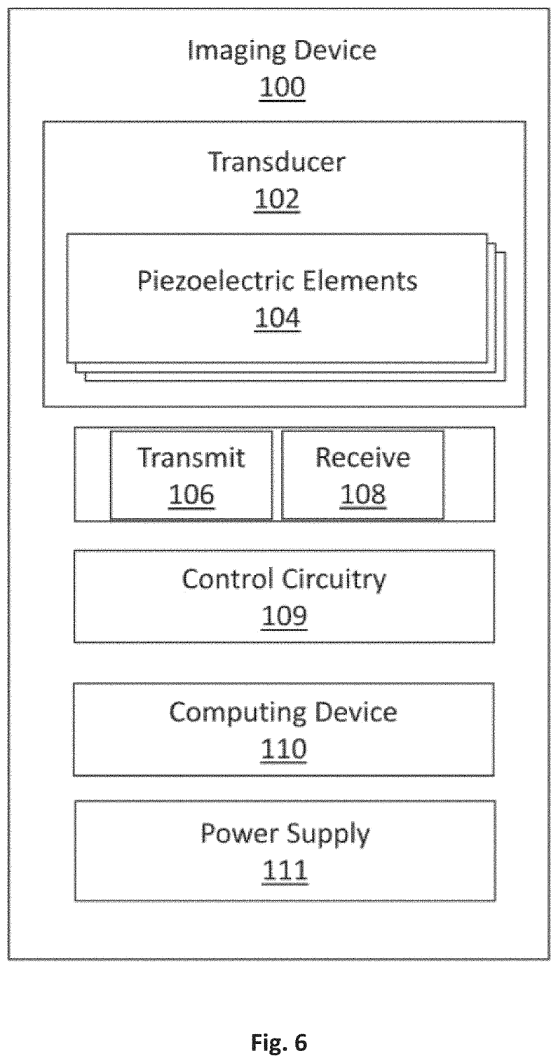

FIG. 6 illustrates a block diagram of an imaging device (100) with transmit (106) and receive channels (108), controlled by control circuitry (109), and a computing device (110) configured to implement various imaging computations. The imaging device (100) may optionally include a power supply (111) to energize the various components of the imaging device (100). In some cases, the computing device (110) and/or the power supply (111) may be external to the imaging device (100).

In some embodiments, the imaging device (100) may be used to generate an image of internal tissue, bones, blood flow, or organs of human or animal bodies. In some cases, the imaging device (100) transmits a signal into the body and receives a reflected signal from the body part being imaged. Such imaging devices (100) may include, for instance, piezoelectric transducers (102), which may also be referred to herein as transceivers or imagers, and which may be based on photo-acoustic or ultrasonic effects. The imaging device (100) can be used to image other objects as well. For example, the imaging device (100) can be used in medical imaging, flow measurements for fluids or gases in pipes, lithotripsy, and localized tissue heating for therapeutic and highly intensive focused ultrasound (HIFU) surgery.

In addition to use with human patients, the imaging device 100 may be used to image internal organs of an animal as well. Moreover, in addition to imaging internal organs, the imaging device 100 may also be used to determine direction and velocity of blood flow in arteries and veins, as well as tissue stiffness, with Doppler mode imaging.

The imaging device 100 may be used to perform different types of imaging. For example, the imaging device 100 may be used to perform one dimensional imaging, also known as A-Scan, 2D imaging, also known as B scan (B-mode), three dimensional (3D) imaging, also known as C scan, and Doppler imaging. The imaging device 100 may be switched to different imaging modes and electronically configured under program control.

To facilitate imaging, the imaging device 100 includes an array of piezoelectric transducers 102, each piezoelectric transducer 102 including an array of piezoelectric elements 104. A piezoelectric element 104 may also include two of more sub-elements, each of which may be configurable in a transmit and/or receive operation. The piezoelectric elements 104 may operate to 1) generate waves (e.g., sound waves or pressure waves) that can pass through the body or other mass and 2) receive reflected waves off the object within the body, or the other mass, to be imaged.

In some examples, the imaging device 100 may be configured to simultaneously transmit and receive ultrasonic waveforms. For example, certain piezoelectric elements 104 may send pressure waves toward the target object being imaged while other piezoelectric elements 104 receive the pressure waves reflected from the target object and develop electrical charges in response to the received waves. The electrical charges may be interpreted and/or processed to generate an image of the target object or a portion thereof.

In some examples, each piezoelectric element 104 may emit or receive signals at a certain frequency, known as a center frequency, as well as the second and/or additional frequencies. Such multi-frequency piezoelectric elements 104 may be referred to as multi-modal piezoelectric elements 104 and can expand the bandwidth of the imaging device 100.

The piezoelectric material that forms the piezoelectric elements 104 may contract and expand when different voltage values at a certain frequency are applied. Accordingly, as voltages alternate between different values applied, the piezoelectric elements 104 may transform the electrical energy (i.e., voltages) into mechanical movements resulting in acoustic energy which is emitted as waves at the desired frequencies. These waves are reflected from a target being imaged and are received at the same piezoelectric elements 104 and converted into electrical signals that are then used to form an image of the target.

To generate the pressure waves, the imaging device 100 may utilize a number of transmit channels 106 and a number of receive channels 108. The transmit channels 106 include a number of components that drive the transducer 102, (i.e., the array of piezoelectric elements 104), with a voltage pulse at a frequency that they are responsive to. This causes an ultrasonic waveform to be emitted from the piezoelectric elements 104 towards an object to be imaged. The ultrasonic waveform travels towards the object to be imaged and a portion of the waveform is reflected back to the transducer 102, where the receive channels 108 collect the reflected waveform, convert it to an electrical energy, and process it, for example, at the computing device 110, to develop an image that can be displayed and interpreted by a human or a computer.

In some examples, while the number of transmit channels 106 and receive channels 108 in the imaging device 100 remain constant, the number of piezoelectric elements 104 that they are coupled to may vary. This coupling can be controlled by control circuitry 109. In some examples, a portion of the control circuitry 109 may be distributed in the transmit channels 106 and in the receive channels 108. For example, the piezoelectric elements 104 of a transducer 102 may be formed into a 2D array with N columns and M rows.

In one example, the 2D array of piezoelectric elements 104 may have a number of columns and rows, such as 128 columns and 32 rows. The imaging device 100 may have up to 128 transmit channels 106 and up to 128 receive channels 108. Each transmit channel 106 and receive channel 108 can be coupled to multiple or single piezoelectric elements or sub-elements 104. Depending on the imaging mode, each column of piezoelectric elements 104 may be coupled to a single transmit channel 106 and a single receive channel 108. The transmit channel 106 and receive channel 108 may receive composite signals, which composite signals combine signals received at each piezoelectric element 104 within a respective row or column. In another example, (i.e., during a different imaging mode), individual piezoelectric elements 104 can be coupled to their own transmit channel 106 and their own receive channel 108.

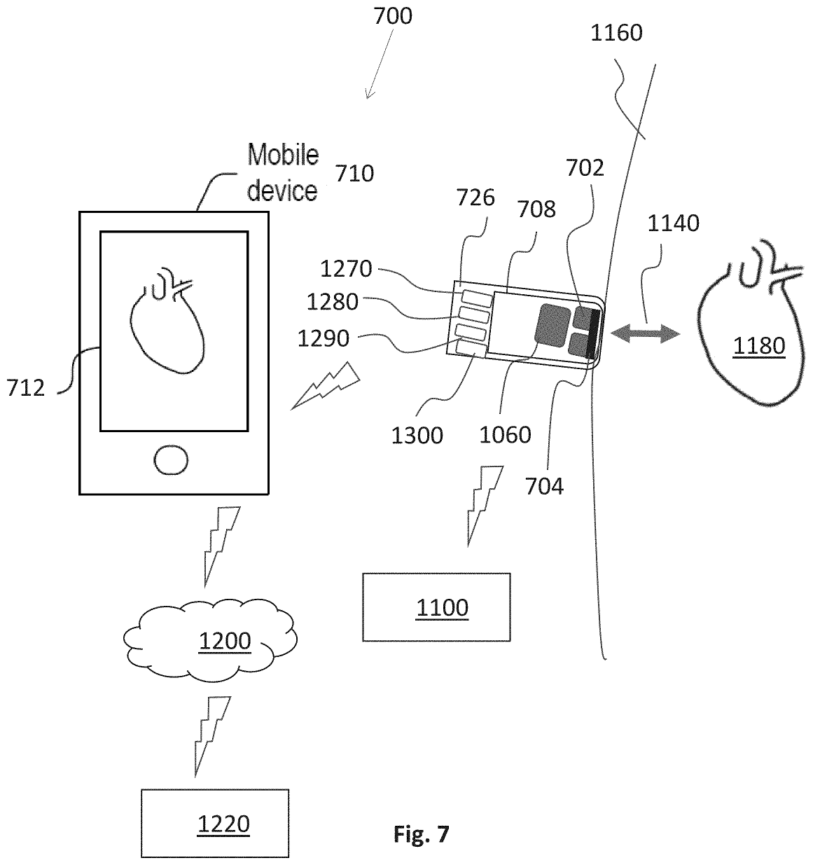

FIG. 7 shows another exemplary embodiment of the ultrasonic imaging system 700 disclosed herein. The imaging system 700 may include a portable device 710 having a display unit 712 and a data recording unit with connection enabled by communication interface to a network 1200, and external databases 1220, such as electronic health records. Such connection to external data sources may facilitate medical billing, data exchange, inquiries, or other medical related information communication. The system 700 may include an ultrasonic imager (interchangeable as “probe” herein) 726 which includes ultrasonic imager assembly (interchangeably as “tile assembly” herein) 708, where the ultrasonic tile has one or more arrays of pMUTs 702 fabricated on a substrate. The array(s) of pMUTs 702 may be configured to emit and receive ultrasonic waveforms.

The pMUT array 702 may be operatively coupled to (i) an application specific integrated circuit (ASIC) 1060 located in close proximity to the pMUT array 702 and/or (ii) another control unit 1100 located remote from the pMUT array 702. The array may be coupled to impedance lowering and/or impedance matching material 704 which can be placed adjacent to the pMUT array. In some embodiments, the imager 726 includes a rechargeable power source 1270 and/or a connection interface 1280 to an external power source, e.g., a USB interface. In some embodiments, the imager 726 includes an input interface 1290 for an ECG signal for synchronizing scans to ECG pulses. In some embodiments, the imager 726 has an inertial sensor 1300 to assist with user guidance.

In some embodiments, many pMUT arrays can be batch manufactured at low cost. Further, integrated circuits can also be designed to have dimensions such that connections needed to communicate with pMUTs are aligned with each other and pMUT array can be connected to a matching integrated circuit in close proximity, typically vertically below or proximal to the array by a distance, e.g., around 25 μm to 100 μm. Larger arrays of pMUT elements can also be achieved by using multiple pMUT arrays, along with multiple matching ASICs and assembling them adjacent to each other and covering them with appropriate amounts of impedance matching material. Alternately, a single array can have large number of pMUT elements arranged in rectangular arrays or other shapes with a number of pMUT elements ranging from less than 1000 to 10,000. The pMUT array and the plurality of pMUT elements can be connected to matching ASICs.

The arrow 1140 shows ultrasonic transmit beams from the imager assembly 708 targeting a body part 1160 and imaging a target 1180. The transmit beams are reflected by the target being imaged and enter the imager assembly 708 as indicated by arrow 1140. In addition to an ASIC 1060, the imaging system 700 may include other electronic control, communication, and computational circuitry 1100. It is understood that the ultrasonic imager 708 can be one self-contained unit, or it may include physically separate, but electrically or wirelessly connected elements, such as the electronic control unit 1100.

In some cases, the ASIC 1060 can comprise one or more low noise amplifiers (LNA). The pMUTs can be connected to the LNA in receive mode through switches. The LNA converts the electrical charge in the pMUT generated by a reflected ultrasonic beam exerting pressure on the pMUT, to an amplified voltage signal with low noise. The signal to noise ratio of the received signal can be among the key factors that determine the quality of the image being reconstructed. It is thus desirable to reduce inherent noise in the LNA itself. This can be achieved by increasing the transconductance of the input stage of the LNA. This can be achieved for example by using more current in the input stage. More current may cause power dissipation and heat to increase. However, in cases where low voltage pMUTs are used, with ASIC in close proximity, the power saved by the low voltage pMUTs can be utilized to lower noise in the LNA for a given total temperature rise acceptable when compared to transducers operated with high voltage.

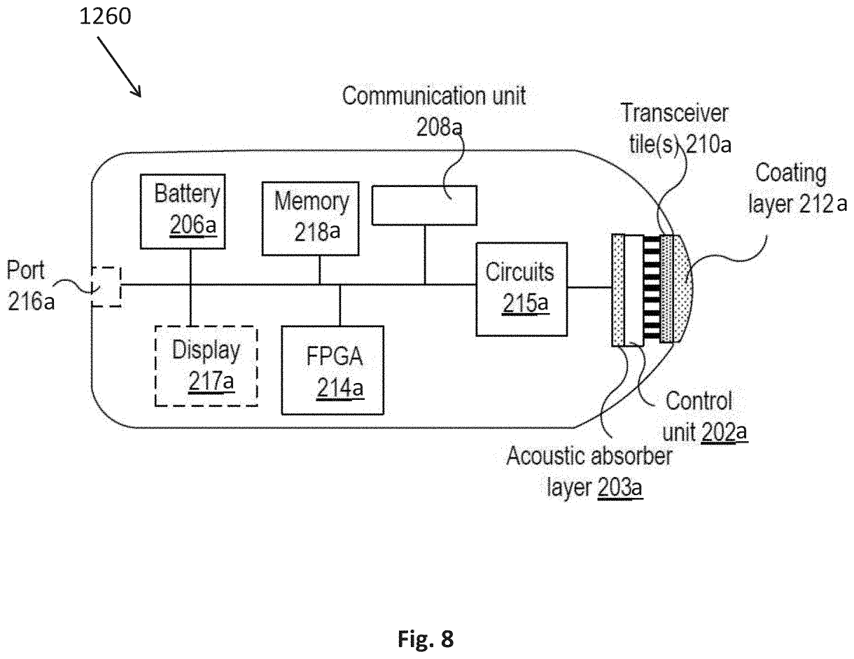

FIG. 8 shows a schematic diagram of another example of an imager 1260. The imager 1260 may include a transceiver array 210a for transmitting and receiving pressure waves and a coating layer 212a that operates as a lens for steering the propagation direction of and/or focusing the pressure waves and also functions as an impedance interface between the transceiver array and the human body. The lens 212a may cause attenuation of the signal exiting the transducer and also entering the transducer. The imager 1260 may also include a control unit 202a, such as an ASIC, for controlling the transceiver array 210a and coupled to the transducer array 210a. The combination of the transceiver array with the ASIC connected to it may constitute a tile. Additional components may include one or more Field Programmable Gate Arrays (FPGAs) 214a for controlling the components of the imager 1260, a circuit(s) 215a, such as Analog Front End (AFE), for processing/conditioning signals; and an acoustic absorber layer 203a for absorbing waves that are generated by the transducer array 210a and propagate toward the circuit 215a. In certain embodiments the acoustic absorber layer can be located behind the ASIC (relative to the transducer being in front of the ASIC). In other embodiments, the acoustic absorber layer can be located in between the transducer and the ASIC. Additional components may optionally include a communication unit 208a for communicating data with an external device through one or more ports 216a; a memory 218a for storing data; a battery 206a for providing a more portable source of electrical power to the components of the imager; and/or a display 217a for displaying a user interface and ultrasound-derived images.

During operation of the imager 1260, a user may cause the pMUTs surface, covered by an interface material, to contact a body part area upon which ultrasonic waves are transmitted towards the target being imaged. The imager receives reflected ultrasonic beams from the imaging target and processes or transmits the reflected beams to an external processor for image processing and/or reconstruction, and then to a portable device for displaying an image.

When using the imager, for example to image human or animal body part, the transmitted ultrasonic waveform can be directed towards the target. Contact with the body can be achieved by holding the imager in close proximity of the body, usually after a gel is applied on the body and the imager placed on the gel, to allow superior interface of ultrasonic waves being emitted to enter the body and also for ultrasonic waveforms reflected from the target to reenter the imager, where the reflected signal is used to create an image of the body part and results displayed on a screen, including graphs, plots, statistics shown with or without the images of the body part in a variety of formats.

In some embodiments, the imager/probe may be configured with certain parts being physically separate yet connected through a cable or wireless communications connection. In one example, the pMUT assembly and the ASIC and some control and communications related electronics can reside in a unit often called a probe. The part of the device or probe that contacts the body part may comprise the pMUT assembly.

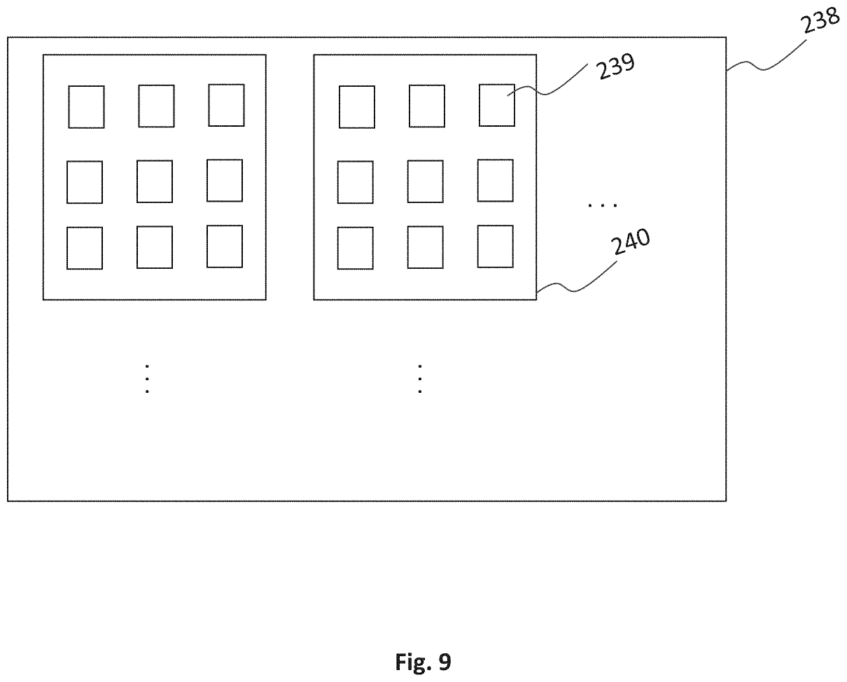

FIG. 9 shows a substrate 238, on which a plurality of piezoelectric micro machined ultrasound transducer (pMUT) array elements 239 can be arranged. One or more array elements may form a transceiver array 240, and more than one transceiver array may be included on the substrate 238. The operation of the individual array elements may be controlled or adjusted using the scan tables described herein.

### Computer Systems

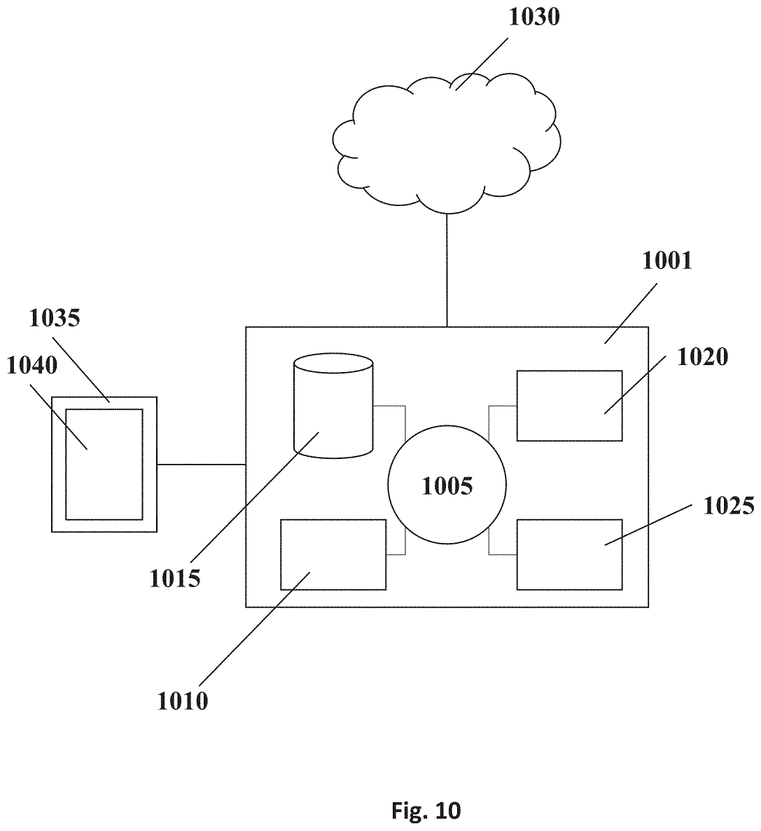

The present disclosure provides computer systems that are programmed to implement methods of the present disclosure. FIG. 10 shows a computer system 1001 that can be programmed or otherwise configured to one or more methods of the present disclosure. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries, and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for controlling the ultrasound imagers and probes described herein, selecting or modifying one or more scan tables, or viewing one or more images obtained using the ultrasound imagers/probes and the scan tables. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1005.

Although certain embodiments and examples are provided in the foregoing description, the instant subject matter extends beyond the specifically disclosed embodiments to other alternative embodiments and/or uses, and to modifications and equivalents thereof. Thus, the scope of the claims appended hereto is not limited by any of the particular embodiments described. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein A and/or B encompasses one or more of A or B, and combinations thereof such as A and B. It will be understood that although the terms “first,” “second,” “third,” etc. may be used herein to describe various elements, components, regions and/or sections, these elements, components, regions and/or sections should not be limited by these terms. These terms are merely used to distinguish one element, component, region or section from another element, component, region, or section. Thus, a first element, component, region, or section discussed below could be termed a second element, component, region, or section without departing from the teachings of the present disclosure.

The terminology used herein is for the purpose of describing particular embodiments only and is not intended to limit the present disclosure. As used herein, the singular forms “a,” “an,” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. A method, comprising:
a. generating a map comprising an index of a plurality of scan tables;
b. imaging a first target region using one or more scan tables selected from the plurality of scan tables;
c. computing a parameter of interest based on one or more images obtained from the imaging in (b);
d. imaging the first target region using a subset of the one or more scan tables, which subset is selected based on the parameter of interest computed in (c).

2. The method of claim 1, wherein the plurality of scan tables are ordered or grouped according to an imaging condition of interest.

3. The method of claim 2, wherein the imaging condition of interest comprises a beam configuration, beam penetration, beam transmit power, imaging frame rate, imaging frequency, scanning line density, signal to noise ratio, or imaging resolution.

4. The method of claim 1, wherein the parameter of interest comprises a beam configuration, beam penetration, beam transmit power, imaging frame rate, imaging frequency, scanning line density, signal to noise ratio, or imaging resolution.

5. The method of claim 1, further comprising, prior to (a), loading a super-set of the plurality of scan tables onto a memory with instructions for executing steps (a)-(d).

6. The method of claim 1, further comprising persisting one or more image frames obtained using a first scan table and a second scan table to show a transition of imaging states or imaging parameters.

7. The method of claim 1, further comprising repeating steps (b)-(d) for a second target region.

8. The method of claim 7, further comprising selecting a different scan table or a different set of scan tables for imaging of the second target region.

9. The method of claim 7, wherein the second target region comprises a different anatomy than the first target region.

10. The method of claim 1, wherein the plurality of scan tables comprise a collection of events and associated scanning conditions or parameters for an imaging device.

11. The method of claim 10, wherein the imaging device comprises an ultrasound or audio-acoustic imaging device.

12. The method of claim 10, wherein the collection of events comprises one or more events comprising a description of a scanning condition or a scanning parameter for an imaging line.

13. The method of claim 12, wherein the scanning condition or scanning parameter comprises a transmit pulse parameter, an aperture parameter, a delay parameter, a filter parameter, a decimation parameter, a line-spacing parameter, or a number of collinear transmits parameter.

14. The method of claim 1, wherein the map enables a dynamic selection of one or more optimal scan tables comprising one or more predetermined sets of operating conditions for one or more imaging events.

15. The method of claim 1, further comprising displaying one or more images of the first target region to a user or an operator of an imaging device used to capture the one or more images.

16. A system, comprising:
an imaging device;
a memory comprising an index of a plurality of scan tables; and
a processor, wherein the processor is configured to:
image a first target region using one or more scan tables selected from the plurality of scan tables;
compute a parameter of interest based on one or more images obtained from the imaging of the first target region; and
image the first target region using a subset of the one or more scan tables, which subset is selected based on the computed parameter of interest.

17. The system of claim 16, wherein the plurality of scan tables are ordered or grouped according to an imaging condition of interest.

18. The system of claim 17, wherein the imaging condition of interest comprises a beam configuration, beam penetration, beam transmit power, imaging frame rate, imaging frequency, scanning line density, signal to noise ratio, or imaging resolution.

19. The system of claim 16, wherein the parameter of interest comprises a beam configuration, beam penetration, beam transmit power, imaging frame rate, imaging frequency, scanning line density, signal to noise ratio, or imaging resolution.

20. The system of claim 16, wherein the processor is further configured to persist one or more image frames obtained using a first scan table and a second scan table to show a transition of imaging states or imaging parameters over time.

21. The system of claim 16, wherein the processor is further configured to image a second target region.

22. The system of claim 21, wherein the processor is further configured to select a different scan table or a different set of scan tables for imaging of the second target region.

23. The system of claim 21, wherein the second target region comprises a different anatomy than the first target region.

24. The system of claim 16, wherein the plurality of scan tables comprise a collection of events and associated scanning conditions or parameters for the imaging device.

25. The system of claim 24, wherein the imaging device comprises an ultrasound or audio-acoustic imaging device.

26. The system of claim 24, wherein the collection of events comprises one or more events comprising a description of a scanning condition or a scanning parameter for an imaging line or one or more imaging operations performable using the imaging device.

27. The system of claim 26, wherein the scanning condition or scanning parameter comprises a transmit pulse parameter, an aperture parameter, a delay parameter, a filter parameter, a decimation parameter, a line-spacing parameter, or a number of collinear transmits parameter.

28. The system of claim 16, wherein the processor is configured to dynamically select one or more optimal scan tables comprising one or more predetermined sets of operating conditions for one or more imaging events.

29. The system of claim 16, further comprising a display unit for displaying the one or more images of the first target region to a user or an operator of the imaging device.

30. The system of claim 16, wherein the plurality of scan tables are loaded onto the memory as a super-set of multiple scan tables.
